(19) United States
(12) Patent Application Publication
(10) Pub. No.: US 2024/0115193 A1
(43) Pub. Date: Apr. 11, 2024

(54) SLEEP QUALITY ASSESSMENT METHOD, DEVICE, AND COMPUTER-READABLE STORAGE MEDIUM

(71) Applicant: BOMDIC INC., New Taipei City (TW)

(72) Inventors: Haoyi Chih, New Taipei City (TW); Chun-Yen Chiang, New Taipei City (TW)

(73) Assignee: BOMDIC INC., New Taipei City (TW)

(21) Appl. No.: 18/193,656

(22) Filed: Mar. 31, 2023

Related U.S. Application Data
(60) Provisional application No. 63/411,135, filed on Sep. 29, 2022.

(51) Int. Cl. A61B 5/00

(52) U.S. Cl. CPC A61B 5/4815; A61B 5/7264; A61B 5/02416

(57) ABSTRACT

An embodiment of the invention provides a sleep quality assessment method, a device, and a computer-readable storage medium. The method includes the following. Multiple sleep indicators associated with a sleep process of a subject are obtained, and a first sleep assessment result of the subject is determined based on the multiple sleep indicators. A sleep signal corresponding to the sleep process is obtained, and a second sleep assessment result of the subject is determined based on the sleep signal. A sleep quality of the sleep process is determined based on the first sleep assessment result and the second sleep assessment result.

### CROSS-REFERENCE TO RELATED APPLICATION

This application claims the priority benefit of U.S. provisional application Ser. No. 63/411,135, filed on Sep. 29, 2022. The entirety of the above-mentioned patent application is hereby incorporated by reference herein and made a part of this specification.

### BACKGROUND

### Technical Field

The invention relates to a sleep analysis mechanism, and particularly relates to a sleep quality assessment method, a device, and a computer-readable storage medium.

### Description of Related Art

A sleep quality score is an indicator used to assess the sleep quality and effectiveness of a person, which are usually measured and analyzed by using sleep monitoring apparatuses, and these apparatuses may track sleep indicators such as the sleep duration, sleep stages, breathing, and heart rate.

However, most existing technologies only determine the sleep quality score based on various sleep indicators, and do not directly refer to sleep signals measured from a sleep process.

### SUMMARY

In view of this, the invention provides a sleep quality assessment method, a device, and a computer-readable storage medium, which may be used to solve the above technical problems.

An embodiment of the invention provides a sleep quality assessment method, suitable for a sleep quality assessment device, which includes the following. A first sleep signal is obtained via a sensor, multiple sleep indicators associated with a sleep process of a subject are determined based on the first sleep signal, and a first sleep assessment result of the subject is determined based on the multiple sleep indicators. A second sleep signal corresponding to the sleep process is obtained via the sensor, and a second sleep assessment result of the subject is determined based on the second sleep signal. A sleep quality of the sleep process is determined based on the first sleep assessment result and the second sleep assessment result.

An embodiment of the invention provides a sleep quality assessment device, including a storage circuit and a processor. The storage circuit stores a program code. The processor is coupled to the storage circuit and accesses the program code to execute the following. A first sleep signal is obtained via a sensor, multiple sleep indicators associated with a sleep process of a subject are determined based on the first sleep signal, and a first sleep assessment result of the subject is determined based on the multiple sleep indicators. A second sleep signal corresponding to the sleep process via the sensor is obtained, and a second sleep assessment result of the subject is determined based on the second sleep signal. A sleep quality of the sleep process is determined based on the first sleep assessment result and the second sleep assessment result.

An embodiment of the invention provides a computer-readable storage medium. The computer-readable storage medium performs recording to an executable computer program, and the executable computer program is loaded by a sleep quality assessment device to execute the following steps. A first sleep signal is obtained via a sensor, multiple sleep indicators associated with a sleep process of a subject are determined based on the first sleep signal, and a first sleep assessment result of the subject is determined based on the multiple sleep indicators. A second sleep signal corresponding to the sleep process is obtained via the sensor, and a second sleep assessment result of the subject is determined based on the second sleep signal. A sleep quality of the sleep process is determined based on the first sleep assessment result and the second sleep assessment result.

### BRIEF DESCRIPTION OF THE DRAWINGS

FIG. 1 illustrates a schematic diagram of a sleep quality assessment device according to an embodiment of the invention.

FIG. 2 illustrates a flowchart of a sleep quality assessment method according to an embodiment of the invention.

FIG. 3 illustrates a schematic diagram of determining a sleep quality score corresponding to each sleep indicator according to an embodiment of the invention.

### DESCRIPTION OF THE EMBODIMENTS

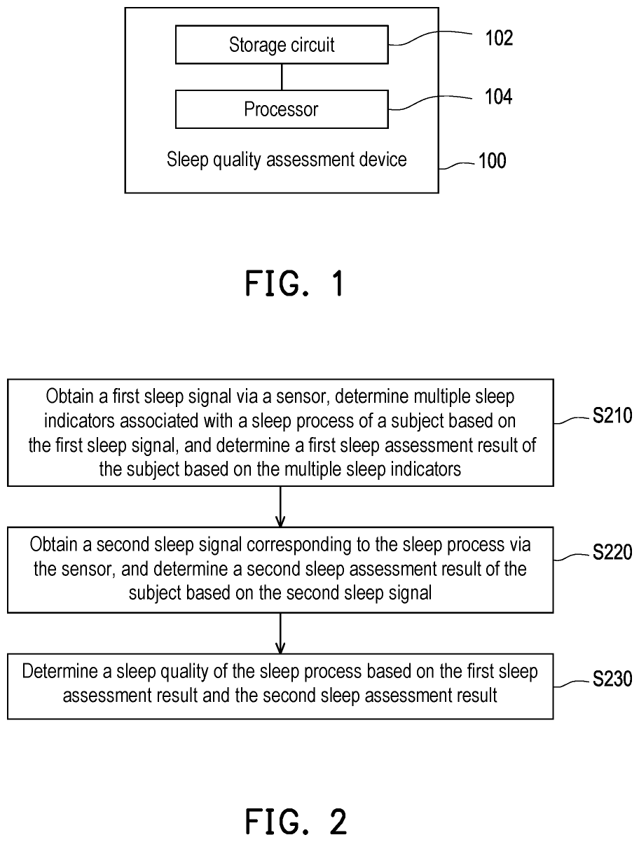

Please refer to FIG. 1, which illustrates a schematic diagram of a sleep quality assessment device according to an embodiment of the invention. In different embodiments, a sleep quality assessment device 100 may be implemented as various smart devices, computer devices, and/or wearable devices (such as smart watches, bracelets, rings), but is not limited thereto.

In FIG. 1, the sleep quality assessment device 100 includes a storage circuit 102 and a processor 104. The storage circuit 102 is, for example, any type of fixed or removable random access memory (RAM), read-only memory (ROM), flash memory, hard disk or other similar device or a combination of these devices, and may be used to record multiple program codes or modules.

The processor 104 is coupled to the storage circuit 102 and may be a general purpose processor, a special purpose processor, a conventional processor, a digital signal processor, multiple microprocessors, one or more microprocessors combined with a digital signal processor core, a controller, a microcontroller, an application specific integrated circuit (ASIC), a field programmable gate array (FPGA), any other kind of ICs, state machines, processors for advanced RISC machines (ARM) and the like.

In an embodiment of the invention, the processor 104 may access the modules and program codes recorded in the storage circuit 102 to implement a sleep quality assessment method proposed by the invention, and the details of which are described below.

Please refer to FIG. 2, which illustrates a flowchart of a sleep quality assessment method according to an embodiment of the invention. The method of this embodiment may be executed by the sleep quality assessment device 100 in FIG. 1, and the details of each step in FIG. 2 will be described below in combination with components in FIG. 1.

First, in Step S210, the processor 104 obtains a first sleep signal via the sensor, multiple sleep indicators associated with a sleep process of a subject (hereinafter referred to as A) are determined based on the first sleep signal, and a first sleep assessment result of the subject is determined based on the multiple sleep indicators.

In some embodiments, the subject A may, for example, wear the sleep quality assessment device 100 (for example, a smart watch) during the sleep process thereof, so that the sleep quality assessment device 100 collects various sleep signals (such as photoplethysmography (PPG) signals, inertial measurement unit (IMU) signals) and/or data (such as the heart rate, the heart rate variability, the respiratory rate, the resting heart rate) of the subject A during the sleep process via the sensor as the first sleep signal. Afterward, the sleep quality assessment device 100 may determine one or more corresponding sleep indicators based on the collected data.

In some embodiments, the subject A may wear a specific smart device (for example, a smart watch) during the sleep process thereof, and the smart device may collect various signals and/or data of the subject A during the sleep process. Afterward, the smart device may determine one or more corresponding sleep indicators based on the collected signals/data, and provide the determined sleep indicators to the sleep quality assessment device 100. Alternatively, the smart device may also provide the collected signals/data to the sleep quality assessment device 100, so that the sleep quality assessment device 100 may determine one or more corresponding sleep indicators, but is not limited thereto.

In different embodiments, the one or more sleep indicators may include a sleep latency, a sleep duration, a wake after sleep onset (WASO), a sleep efficiency, a proportion of rapid eye movement period, a proportion of falling asleep period (commonly known as an N1 period), a proportion of light sleep period (commonly known as an N2 period), and a proportion of deep sleep period (commonly known as an N3 period), but is not limited thereto.

In the embodiment of the invention, how to determine the sleep indicator based on the signal/data collected during the sleep process may refer to related art documents to select a suitable determination method, which will not be repeated here.

In an embodiment, in a process of determining the first sleep assessment result of the subject based on the multiple sleep indicators, the processor 104 may obtain a sleep quality score corresponding to each sleep indicator, and determine the first sleep assessment result based on the sleep quality score corresponding to each sleep indicator.

In the embodiment of the invention, methods of determining the sleep quality scores corresponding to respective sleep indicators are similar, so one of the sleep indicators (hereinafter referred to as a first sleep indicator) is taken as an example for illustration below.

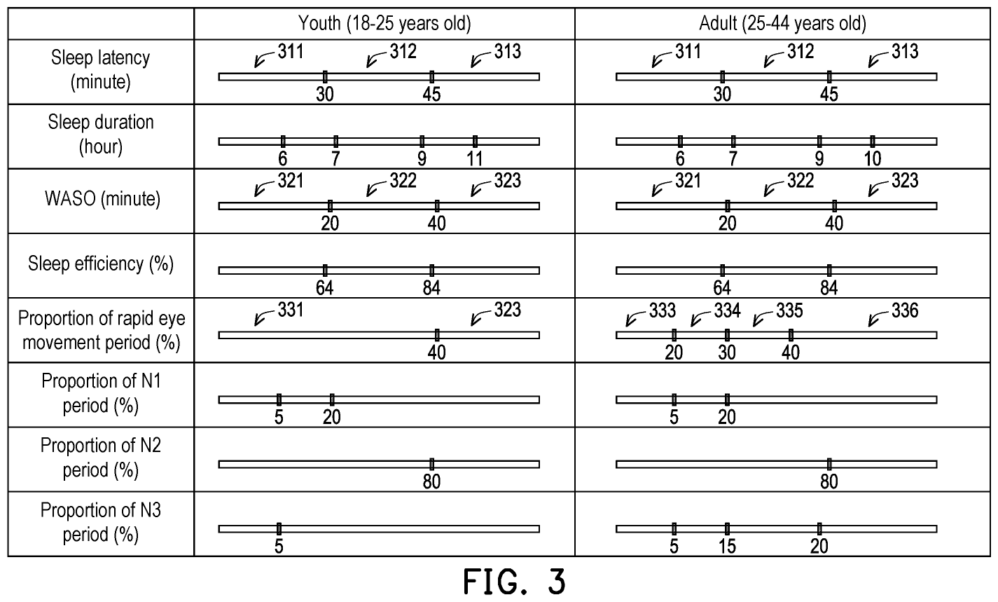

In an embodiment, the first sleep indicator may be determined to have a first numerical result. In this case, the processor 104 may obtain multiple numerical ranges corresponding to the first sleep indicator, and determine which numerical range the first numerical result is in among the multiple numerical ranges. The processor 104 may use a score corresponding to a first numerical range as the sleep quality score corresponding to the first sleep indicator in response to determining that the first numerical result is in the first numerical range among the multiple numerical ranges. In order to make the above concepts easier to understand, the following is supplemented with FIG. 3 for further description.

Please refer to FIG. 3, which illustrates a schematic diagram of determining a sleep quality score corresponding to each sleep indicator according to an embodiment of the invention.

In the scenario of FIG. 3, assuming that the first sleep indicator considered is the sleep latency, then the processor 104 may correspondingly obtain a numerical range 311-313 corresponding to the sleep latency, in which the numerical range 311-313 may be respectively assigned with corresponding scores. For example, the scores corresponding to the numerical range 311-313 are, for example, 3, 2, and 1, respectively, but is not limited thereto. In this case, assuming that the sleep latency corresponding to the sleep process of the subject A is determined to be 35 minutes, the processor 104 may regard it as the first numerical result, and accordingly determine that the first numerical result is in which of the numerical range 311-313. In this embodiment, the processor 104 may determine, for example, that the first numerical result is in the numerical range 312 (which is between 30 minutes and 45 minutes), and use the score corresponding to the numerical range 312 (for example, 2) as the sleep quality score corresponding to the sleep latency.

In addition, assuming that the first sleep indicator considered is the WASO, the scores corresponding to the numerical range 321-323 corresponding to the WASO are, for example, 6, 5, and 4, respectively, but is not limited thereto. In this case, assuming that the WASO corresponding to the sleep process of the subject A is determined to be 30 minutes, which may be regarded as the first numerical result, and is accordingly determined that the first numerical result is in the numerical range 322 (which is between 20 minutes and 40 minutes), and the score corresponding to the numerical range 322 (for example, 5) is used as the sleep quality score corresponding to the WASO.

In an embodiment, when obtaining multiple numerical ranges corresponding to the first sleep indicator, the processor 104 may determine the multiple numerical ranges corresponding to the first sleep indicator based on an age of the subject A.

Taking the scenario in FIG. 3 as an example again, assuming that the first sleep indicator considered is the proportion of rapid eye movement period, the processor 104 may consider selecting the numerical range 331 and 332 corresponding to youths based on the age of the subject A, or select the numerical range 333-336 corresponding to adults.

For example, if the age of the subject A (for example, 20 years old) is determined to be young, the processor 104 may select the numerical range 331 and 332 accordingly, and the corresponding scores are, for example, 4 and 2, but is not limited thereto. In this case, assuming that the proportion of rapid eye movement period corresponding to the sleep process of the subject A is determined to be 30%, which may be regarded as the first numerical result, and the first numerical result may be accordingly determined to be in the numerical range 331 (between 0% to 40%), and the score corresponding to the numerical range 331 (for example, 4) is used as the sleep quality score corresponding to the proportion of rapid eye movement period.

For another example, if the age of the subject A (for example, 40 years old) is determined to be an adult, the processor 104 may select the numerical range 333-336 accordingly, and assign corresponding scores such as 3, 4, 3, and 2, but is not limited thereto. In this case, assuming that the proportion of rapid eye movement corresponding to the sleep process of the subject A is determined to be 35%, which may be regarded as the first numerical result, and the first numerical result may be accordingly determined to be in the numerical range 335 (between 30% to 40%), and the score corresponding to the numerical range 335 (for example, 3) is used as the sleep quality score corresponding to the proportion of rapid eye movement period.

In the embodiment of the invention, the sleep quality scores corresponding to other sleep indicators should be deduced according to the above teachings, and will not be repeated here.

In an embodiment, after obtaining the sleep quality scores corresponding to each sleep indicator, the processor 104 may determine a first sleep score (hereinafter referred to as SC1) as the first sleep assessment result by executing a linear combination on the sleep quality score corresponding to each sleep indicator.

In an embodiment, the first sleep score SC1 may be represented as:

$$\frac{β SL + β TST + β WASO + β REM + β N 3 + β SE}{18} \times 100.$$

in which SL is a score corresponding to the sleep latency, TST is a score corresponding to the total sleep time (that is, a sum of a sleep period and the WASO), REM is a score corresponding to the proportion of rapid eye movement period, N3 is a score corresponding to the N3 period, SE is a score corresponding to the sleep efficiency, and β is a constant (which may be set to any numerical according to the requirement of a designer, such as 1).

In the embodiment of the invention, since the first sleep score SC1 is determined based on multiple sleep indicators, which not only has a high degree of reliability, but also provides a certain degree of interpretability. For example, when the first sleep score SC1 is low, reasons for the low first sleep score SC1 (such as a long WASO and/or a long sleep latency) may be found among the considered sleep indicators, but is not limited thereto.

Please refer to FIG. 2 again. In Step S220, the processor 104 obtains a second sleep signal corresponding to the sleep process via the sensor, and determines a second sleep assessment result of the subject according to the second sleep signal.

In different embodiments, the second sleep signal is, for example, the previously mentioned signal (for example, the PPG signal) collected via the sensor during the sleep process of the subject A, but is not limited thereto.

In an embodiment, the processor 104 may feed the obtained second sleep signal into a machine learning model (hereinafter referred to as M), in which the machine learning model M may generate a second sleep score (hereinafter referred to as SC2) as the second sleep assessment result in response to the second sleep signal. In different embodiments, the machine learning model M may be implemented as a linear regression model, a support vector regression model, and/or various neural networks, etc., but is not limited thereto.

In an embodiment, in order to enable the machine learning model M to have the above-mentioned capabilities, during a training process of the machine learning model M, the designer may feed specially designed training data into the machine learning model M, so that the machine learning model M may perform learning correspondingly. For example, after obtaining a sleep signal (hereinafter referred to as S1) of a sleep process of a user, the user may be asked to self-assess a sleep score (hereinafter referred to as S2) of the sleep process, and mark the sleep signal S1 as corresponding to the sleep score S2. Afterward, the processor 104 may feed the sleep signal S1 and the corresponding sleep score S2 as a piece of training data into the machine learning model M under training, so that the machine learning model M may learn which features the sleep signal S1 corresponding to the sleep score S2 has.

Based on similar concepts, after the sleep signals corresponding to different sleep scores are fed into the machine learning model M under training, the machine learning model M may learn the features of the sleep signals corresponding to different sleep scores. Based on this, when an unknown sleep signal is fed into the trained machine learning model M, the machine learning model M may correspondingly predict/determine the corresponding sleep score, but is not limited thereto.

In the embodiment of the invention, since the second sleep score SC2 is determined by the machine learning model M identifying the features of the second sleep signal of the subject A, in addition to taking into consideration less common sleep features, the determined second sleep score SC2 also has a high correlation with the sleep quality of the subject A itself.

After obtaining the first sleep assessment result and the second sleep assessment result, in Step S230, the processor 104 determines the sleep quality of the sleep process based on the first sleep assessment result and the second sleep assessment result.

In the embodiment in which the first sleep assessment result and the second sleep assessment result are represented as the first sleep score SC1 and the second sleep score SC2 respectively, the processor 104 may, for example, determine a specific sleep score (hereinafter referred to as SC) as the sleep quality of the sleep process by executing a weighted operation on the first sleep score SC1 and the second sleep score SC2.

In an embodiment, before executing the weighted operation, the processor 104 may first scale the first sleep score SC1 and the second sleep score SC2 to the same order of magnitude. For example, both the scaled first sleep score SC1 and the second sleep score SC2 may be numerical values between 0 and 100, but is not limited thereto.

In an embodiment, the first sleep score SC1 and the second sleep score SC2 are respectively configured with a first weight (hereinafter referred to as W1) and a second weight (hereinafter referred to as W2) for executing the weighted operation, in which the specific sleep score SC may be represented as “SC=SC1*W1+SC2*W2”, but is not limited thereto.

In different embodiments, the first weight W1 and the second weight W2 may be set to be any numerical according to the requirement of the designer. For example, the first weight W1 and the second weight W2 may be set to 0.5, but is not limited thereto.

In an embodiment, before executing the weighted operation on the first sleep score SC1 and the second sleep score SC2, the processor 104 may first determine whether the first sleep score SC1 is lower than a preset threshold value (which may be determined by the designer according to requirements). If so, the processor 104 may increase the first weight W1 and decrease the second weight W2, in which the increased first weight W1 may be higher than the decreased second weight W2. That is, when the first sleep score SC1 is low, the processor 104 may increase the weight of the first sleep score SC1 in the process of determining the specific sleep score SC.

In detail, since the first sleep score SC1 is determined based on one or more considered sleep indicators, when the first sleep score SC1 is low (for example, lower than the preset threshold value), the corresponding sleep indicators may also show a situation of poor performance.

At this time, if the specific sleep score SC is determined without particularly increasing the first weight W1 and decreasing the second weight W2, then when the second sleep score SC2 is high, the subject A may see that the considered sleep indicator performs poorly, but the final specific sleep score SC is still high. In this case, the subject A may feel confused instead.

In contrast, if the processor 104 may correspondingly increase the first weight W1 and decrease the second weight W2 when determining that the first sleep score SC1 is low, the generated specific sleep score SC is in line with the performance of the considered sleep indicators, so as to avoid situations that would confuse the subject A or other related personnel.

In some embodiments, the sleep indicator, the first sleep assessment result, the second sleep assessment result, the sleep signal and/or the sleep quality of the sleep process may all be presented visually by a user interface by the sleep quality assessment device 100 or other related electronic devices for reference by the subject A and/or related personnel, but is not limited thereto.

In addition, the invention further provides a computer-readable storage medium for executing the sleep quality assessment method. The computer-readable storage medium comprises multiple program commands (e.g., setup program commands and deploy program commands) implemented therein. The multiple program commands may be loaded into the sleep quality assessment device 100 and executed by the sleep quality assessment device 100 to execute the sleep quality assessment method and functions of the sleep quality assessment device 100.

In summary, the method proposed by the embodiment of the invention may comprehensively consider one or more sleep indicators of the subject and the sleep signal measured during the sleep process to determine the sleep quality of the sleep process. Thereby, the sleep quality of the subject can be determined in a more diverse and comprehensive manner.

Although the invention has been disclosed above with the embodiments, the embodiments are not intended to limit the invention. Persons with ordinary knowledge in the technical field may make some changes and modifications without departing from the spirit and scope of the invention. The scope of protection of the invention should be defined by the appended claims.

What is claimed is:

1. A sleep quality assessment method, suitable for a sleep quality assessment device, comprising:
obtaining a first sleep signal via a sensor, determining a plurality of sleep indicators associated with a sleep process of a subject based on the first sleep signal, and determining a first sleep assessment result of the subject based on the plurality of sleep indicators;
obtaining a second sleep signal corresponding to the sleep process via the sensor, and determining a second sleep assessment result of the subject based on the second sleep signal; and
determining a sleep quality of the sleep process based on the first sleep assessment result and the second sleep assessment result.

2. The method according to claim 1, wherein the plurality of sleep indicators comprise at least one of a sleep latency, a sleep duration, a wake after sleep onset (WASO), a sleep efficiency, a proportion of rapid eye movement period, a proportion of falling asleep period, a proportion of light sleep period, and a proportion of deep sleep period.

3. The method according to claim 1, wherein the step of determining the first sleep assessment result of the subject based on the plurality of sleep indicators comprises:
obtaining a sleep quality score corresponding to each of the plurality of sleep indicators; and
determining the first sleep assessment result based on the sleep quality score corresponding to each of the plurality of sleep indicators.

4. The method according to claim 3, wherein the plurality of sleep indicators comprise a first sleep indicator, the first sleep indicator is determined to have a first numerical result, and the step of obtaining the sleep quality score corresponding to each of the plurality of sleep indicators comprises:
obtaining a plurality of numerical ranges corresponding to the first sleep indicator; and
using a score corresponding to a first numerical range as the sleep quality score corresponding to the first sleep indicator in response to determining that the first numerical result is in the first numerical range among the plurality of numerical ranges.

5. The method according to claim 4, wherein the step of obtaining the plurality of numerical ranges corresponding to the first sleep indicator comprises:
determining the plurality of numerical ranges corresponding to the first sleep indicator based on an age of the subject.

6. The method according to claim 3, wherein the step of determining the first sleep assessment result based on the sleep quality score corresponding to each of the plurality of sleep indicators comprises:
determining a first sleep score as the first sleep assessment result by executing a linear combination on the sleep quality score corresponding to each of the plurality of sleep indicators.

7. The method according to claim 1, wherein the step of determining the second sleep assessment result of the subject based on the second sleep signal comprises:
feeding the second sleep signal into a machine learning model, wherein the machine learning model generates a second sleep score as the second sleep assessment result in response to the second sleep signal.

8. The method according to claim 1, wherein the first sleep assessment result and the second sleep assessment result are represented as a first sleep score and a second sleep score respectively, and the step of determining the sleep quality of the sleep process based on the first sleep assessment result and the second sleep assessment result comprises:
determining a specific sleep score as the sleep quality of the sleep process by executing a weighted operation on the first sleep score and the second sleep score.

9. The method according to claim 8, wherein the first sleep score and the second sleep score are respectively configured with a first weight and a second weight for executing a weighted operation, and before the step of determining the specific sleep score as the sleep quality of the sleep process by executing the weighted operation on the first sleep score and the second sleep score, the method further comprises:
increasing the first weight and decreasing the second weight in response to determining that the first sleep score is lower than a preset threshold value; and
maintaining the first weight and the second weight in response to determining that the first sleep score is not lower than the preset threshold value.

10. The method according to claim 9, wherein the increased first weight is higher than the decreased second weight.

11. A sleep quality assessment device, comprising:
a storage circuit, which stores a program code; and
a processor, which is coupled to the storage circuit and accesses the program code to execute:
obtaining a first sleep signal via a sensor, determining a plurality of sleep indicators associated with a sleep process of a subject based on the first sleep signal, and determining a first sleep assessment result of the subject based on the plurality of sleep indicators;
obtaining a second sleep signal corresponding to the sleep process via the sensor, and determining a second sleep assessment result of the subject based on the second sleep signal; and
determining a sleep quality of the sleep process is based on the first sleep assessment result and the second sleep assessment result.

12. A computer-readable storage medium, the computer-readable storage medium performs recording to an executable computer program, and the executable computer program is loaded by a sleep quality assessment device to execute the following steps:
obtaining a first sleep signal via a sensor, determining a plurality of sleep indicators associated with a sleep process of a subject based on the first sleep signal, and determining a first sleep assessment result of the subject based on the plurality of sleep indicators;
obtaining a second sleep signal corresponding to the sleep process via the sensor, and determining a second sleep assessment result of the subject based on the second sleep signal; and
determining a sleep quality of the sleep process is based on the first sleep assessment result and the second sleep assessment result.
